(19) United States
(12) Patent Application Publication
(10) Pub. No.: US 2023/0083530 A1
(43) Pub. Date: Mar. 16, 2023

(54) DISINFECTION SYSTEM

(71) Applicant: AVALON BIOMEDICAL (MANAGEMENT) LIMITED, HONG KONG (CN)

(72) Inventors: Johnny Yat Ming Chan, Hong Kong (CN); Wai Hong Ho, Hong Kong (CN); Johnson Yiu-Nam Lau, Houston, TX (US)

(73) Assignee: AVALON BIOMEDICAL (MANAGEMENT) LIMITED, HONGKONG (CN)

(21) Appl. No.: 17/795,529

(22) PCT Filed: Nov. 28, 2020
(86) PCT No.: PCT/US2020/070826

§ 371 Date: Jul. 26, 2022

Related U.S. Application Data
(63) Continuation of application No. 29/760,014, filed on Nov. 27, 2020.

(51) Int. Cl. A61L 2/18

(52) U.S. Cl. CPC A61L 2/186; A61L 2202/11; A61L 2202/15; A61L 2202/16

(57) ABSTRACT

A disinfection compartment comprising a plurality of wheels; a casing disposed on the disinfection compartment, at least one tank configured to hold antimicrobial solution or hydrogen peroxide solution, a nozzle/dispenser disposed on the casing, and an attachment member configured to provide a detachably secure attachment to a moving unit, wherein the nozzle/dispenser is an electrostatic sprayer or electrostatic gun.

### CROSS-REFERENCE TO RELATED APPLICATION

This application claims priority to a US design application with the United States Patent and Trademark Office Ser. No. 29/760,014 filed on Nov. 27, 2020; a Hong Kong patent application serial number 32020005636.6 filed on Apr. 9, 2020; and a U.S. provisional patent application with the United States Patent and Trademark Office Ser. No. 62/965,987 filed on Jan. 26, 2020, whose disclosures are incorporated by reference in their entirety herein.

### FIELD OF INVENTION

This invention generally relates to a disinfection system. More particularly, aspects of the invention relate to a system and method for controlling autonomous or remote-controlled mobile robots, in particular, for controlling autonomous or remote-controlled mobile robots using at least one hydrogen peroxide probe.

### BACKGROUND

Among other applications, hydrogen peroxide is used as a disinfectant. It may be used to treat inflammation of the gums and to disinfect (drinking) water. It may also be used to combat excessive microbial growth in water systems and cooling towers. Hydrogen peroxide has been used a first-aid antiseptic for injured skin since the 1920s.

In the United States, hydrogen peroxide has been used more and more frequently to treat individual water supplies. For example, it is used to prevent the formation of colors, tastes, corrosion and scaling by pollution degradation (iron, manganese, sulphates) and micro-organism degradation. Hydrogen peroxide reacts very fast. It will then disintegrate into hydrogen and water, without the formation of byproducts or residuals. This increases the amount of oxygen in the water.

The disinfection mechanism of hydrogen peroxide is based on the release of free oxygen radicals and the pollutants are decomposed by free oxygen radicals. Upon decomposition, only water remains. Free oxygen radicals have both oxidizing and disinfecting abilities. Hydrogen peroxide preforms its disinfecting abilities by damaging, breaking down, misfolding or mutating proteins through oxidation.

Vaporized hydrogen peroxide (VHP) or hydrogen peroxide vapor (HPV) is a vapor form of the hydrogen peroxide. It may be used as an antimicrobial vapor to decontaminate.

VHP is recognized by multiple national agencies including CDC, EPA and NHS as a legitimate antimicrobial pesticide. It may be used as a sanitizer, disinfectant, or sterilant.

### SUMMARY

Since the VHP uses chemical reaction (oxidation) to destroy virus and bacteria, controlling the volume of the VHP applied become the most criterial factor of the efficacy of the disinfection: higher volume of hydrogen peroxide emitted, higher the disinfection efficacy it will be. Additionally, the efficacy is also affected by the ambient temperature, atmosphere pressure and the related humidity.

In the light of the foregoing background, aspects of the invention provide an alternative design of the disinfection system to provide a superior disinfection experience through an effective and accurate yet cost-efficient feedback control.

Accordingly, embodiments of the present invention, in one aspect, may be a disinfection system for disinfecting an enclosed area of a public transport or facility that may include a remote computer system, a probe comprising a color chart, an optical sensor and a radio-frequency module configured to connect to the remote computer system, and a mobile robot comprising a vaporizer configured to vaporize hydrogen peroxide.

Embodiments of the present invention, in another aspect, may be a disinfection compartment comprising a plurality of wheels; a casing disposed on the disinfection compartment; at least one tank configured to hold antimicrobial solution or hydrogen peroxide solution; a nozzle/dispenser disposed on the casing; and an attachment member configured to provide a detachably secure attachment to a moving unit, wherein the nozzle/dispenser is an electrostatic sprayer or electrostatic gun, wherein the disinfection compartment does not include any driving unit to drive its wheels and cannot move itself.

### BRIEF DESCRIPTION OF DRAWINGS

Persons of ordinary skill in the art may appreciate that elements in the figures are illustrated for simplicity and clarity so not all connections and options have been shown. For example, common but well-understood elements that are useful or necessary in a commercially feasible embodiment may often not be depicted in order to facilitate a less obstructed view of these various embodiments of the present disclosure. It may be further appreciated that certain actions and/or steps may be described or depicted in a particular order of occurrence while those skilled in the art may understand that such specificity with respect to sequence is not actually required. It may also be understood that the terms and expressions used herein may be defined with respect to their corresponding respective areas of inquiry and study except where specific meanings have otherwise been set forth herein.

FIG. 1 is a block diagram of a robotic disinfection system according to one embodiment.

FIG. 2 depicts a diagram of an example communication network according to one embodiment.

FIG. 3 is a schematic view of an example of a probe according to one embodiment.

FIG. 4 is a schematic top view of an example of a chemical indicator and a color chart according to one embodiment.

FIG. 5 is a schematic view of a mobile robot according to one embodiment.

FIG. 6 is a schematic view of an example of a mobile robot according to one embodiment.

FIG. 7 is a schematic view of another example of a mobile robot according to one embodiment.

FIG. 8 is flow chart depicting a process to perform concentration measurement based on the operations of a probe according to one embodiment.

FIG. 9 is flow chart depicting a process to perform a recommended action based on an exemplary operation of a mobile robot according to one embodiment.

FIG. 10 is flow chart depicting a process to perform a recommended action based on another example operations of a mobile robot according to one embodiment.

FIG. 11 is an exemplary illustration of an exterior design of a mobile robot (weight: 70 kg; power supply: lithium battery; disinfection method: HPV; Volume for liquefied hydrogen peroxide: 8 L; HPV concentration: 100-200 ppm; disinfection power: 1,000-1,500 m³; continuous working time: 5 hours) according to one embodiment.

FIG. 12 is an exemplary illustration of a disinfection system of the mobile robot according to one embodiment (disinfection in the train; the color of the chemical indicators changes from blue to red after the exposure of HPV at 150 ppm for 15 minutes).

FIGS. 13A-B are illustrations showing the color according to one embodiment (FIG. 13A: disinfection in the train; testing conditions: exposed under HPV (200 ppm) for 1.5 hours; the chemical indicators attached to the ceiling of the train cabin; and colors change in all chemical indicators indicating HPV reaching all the testing area. FIG. 13B: conclusion: according to earlier reports on the disinfection condition (120 ppm for 1 hour), our HPV disinfection procedure (200 ppm for 1.5 hour) should kill enveloped and non-enveloped viruses (including coronavirus) (4-log reduction); according to handheld HPV detector and hydrogen peroxide chemical indicators, the concentration of the hydrogen peroxide reaches 200 ppm at real world disinfection testing).

FIG. 14 is an illustration of a navigation of an interior space within a passenger car according to one embodiment.

FIG. 15 is another illustration of a navigation of an interior space of an airplane cabin or a high speed rail train car according to one embodiment.

FIG. 16 is an exemplary illustration of a compartment configured to detachably dock to a moving unit to form a mobile robot according to one embodiment.

FIG. 17 is a top view of the compartment of FIG. 16.

FIG. 18 is a bottom view of the compartment of FIG. 16.

FIG. 19 is a back view of the compartment of FIG. 16.

FIG. 20 is an illustration of the compartment of FIG. 16 with a detachable mount detached therefrom.

FIG. 21 is a top view of the compartment of FIG. 16 with a detachable mount detached therefrom.

FIG. 22 is a bottom view of the compartment of FIG. 16 with a detachable mount detached therefrom.

FIG. 23 is a back view of the compartment of FIG. 16 with a detachable mount detached therefrom.

FIG. 24 is an illustration of a detachable mount according to one embodiment.

FIG. 25 is a top view of the detachable mount of FIG. 24.

FIG. 26 is a bottom view of the detachable mount of FIG. 24.

FIG. 27 is a front view of the detachable mount of FIG. 24.

FIG. 28 is a back view of the detachable mount of FIG. 24.

FIG. 29 is a side view of the detachable mount of FIG. 24.

### DETAILED DESCRIPTION

Embodiments may now be described more fully with reference to the accompanying drawings, which form a part hereof, and which show, by way of illustration, specific exemplary embodiments which may be practiced. These illustrations and exemplary embodiments may be presented with the understanding that the present disclosure is an exemplification of the principles of one or more embodiments and may not be intended to limit any one of the embodiments illustrated. Embodiments may be embodied in many different forms and should not be construed as limited to the embodiments set forth herein; rather, these embodiments are provided so that this disclosure may be thorough and complete, and may fully convey the scope of embodiments to those skilled in the art. Among other things, the present invention may be embodied as methods, systems, computer readable media, apparatuses, or devices. Accordingly, the present invention may take the form of an entirely hardware embodiment, an entirely software embodiment, or an embodiment combining software and hardware aspects. The following detailed description may, therefore, not to be taken in a limiting sense.

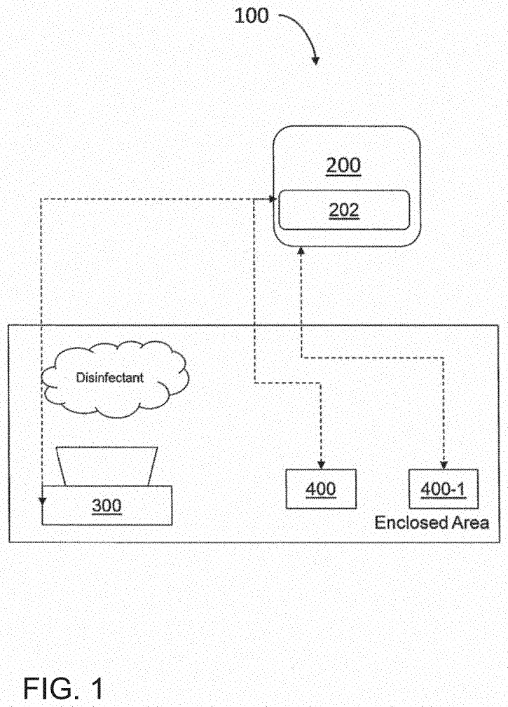

Referring to FIG. 1, a robotic disinfection system 100 comprises a remote computer system 200 that may exchange data with a mobile robot 300 and control it according to parameters that a user directs toward a control application 202. In one embodiment, the control application 202 may be mobile-based app or application. In another embodiment, the control application 202 may be a software program installed with the remote computer system 200. Similarly, the remote computer system 200 may also control and exchange data with at least one probe 400, 400-1. For example, the remote computer system 200 may include a control with software modules that may send and receive signals to/from the probe 400. The remote computer system 200 may include a microprocessor (not shown) and a computer-readable storage medium or memory (not shown) connected to the microprocessor for storing software and data. The mobile robot 300 may include ports, portals, or emitters that may disperse or emit disinfectant to the surrounding environment in response to trigger signals from the control application 202, the remote computer system 200, a combination thereof, or a sensed event on the mobile robot 300. The probe 400 may measure a concentration of the disinfectant around it with sensors and send the collected data to the remote computer system 200. In one embodiment, the probe 400 may send the collected data to the mobile robot 300.

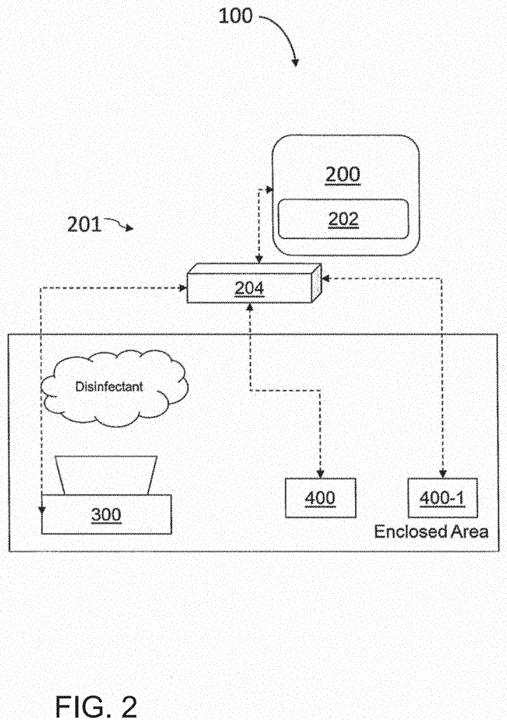

FIG. 2 depicts a diagram of an example communication network 201 according to one embodiment. The mobile robot 300 and the probes 400 may be both wirelessly linked to the remote computer system 200 through a WI-FI hotspot network 204 or other protocols to enable communication between the remote computer system 200, the mobile robot 300 and the probes 400. As such, the mobile robot 300 may include a communications module (not shown) to carry out such communications. In addition, the mobile robot 300 may include a control unit, such as a microprocessor or the like, to control or manage data processing. In another embodiment, the onboard microprocessor of the mobile robot 300 may be configured to analyze images from the probe 400 and operate the mobile robot 300 with or without the assistance from the remote computer system 200. In yet another embodiment, the onboard microprocessor of the mobile robot 300 may further be in an automated mode so that the onboard microprocessor may control the mobile robot 300 to complete the disinfection operation without additional instructions from the operator. In other words, once data of a disinfection plan, parameters of the environment to be disinfected, or other information is downloaded or input into the remote computer system 200 or the onboard microprocessor, the mobile robot 300 may automate its operations or movements to complete tasks for disinfection.

In another embodiment, the communication network 201 may enable the remote computer system 200 to direct command signals to control operations of the mobile robot 300. Similarly, the communication network 201 also may enable the remote computer system 200 to receive collected data from the probes 400 and perform data analysis thereon. In one example, the user may operate the control application 202 or the remote computer system 200 to input operation parameters for a disinfection operation, and the remote computer system 200 may generate a command signal (caused or triggered by the control application 202) to cause the disinfection operation of the mobile robot 300.

For example, the user may configure the control application 202 to select predetermined parameters for different levels of settings, such as public transportation settings, indoor settings, underground settings, etc. In the public transportation settings, the parameters may include parameters such as the length of each cart/car, the width of each cart/car, the height of each cart/car, how many cart/car, model(s) of each cart/car. In another example, there may be sub-parameters or further configurations. For example, the model of each cart/car parameter may provide additional parameters, such as a number of doors, car-to-car connection time, a number of seats, a number of priority seating, a number of handicap spaces, a number of bike allowed, etc. In another embodiment, the control application 202 may receive the parameters from a batch file or a configuration file that may populate a user interface with all the parameters. These parameters may enable the control application 202 to calculate or determine the amount of space/volume needed for the HPVA/HP, the amount of the time for the disinfection, the amount of power needed to drive the mobile robot 300, etc.

In another embodiment, the user may provide parameters on the control application 202 ad-hoc so that the system 100 may be flexible to the settings or environments needed to be disinfected.

In certain examples, the communication network 201 may include a WI-FI hotspot network 204 hosted by the environment or the mobile robot 300. In another embodiment, the communication network 201 may include a combination of the WI-FI hotspot network 204 and a cellular network. In a further embodiment, the communication network 201 may include satellite communication. In yet another example, the communication network 201 may include BLUETOOTH, NFC, or other short-range wireless communication protocols where the mobile robot 300 may communicate with another mobile robot 300. In yet another example, the remote computer system 200 may communicate with the mobile robot 300 and at least one probe 400 through any wireless and/or wired communication protocols.

In some cases, the remote computer system 200 may communicate with a cloud server platform (not shown). For example, via a communication channel, whether via WI-FI (e.g., a wireless connection) or via a wired connection, the remote computer system 200 may upload data collected during the disinfection operation to the cloud server platform. The cloud server platform may execute analysis software to enable the user to analyze the raw data collected. The cloud server platform further may produce and create operation reports based on the collected data. In yet another example, the user or operator of the remote computer system 200 may provide scheduled disinfection plan to the cloud server platform and such disinfection plan may be downloaded to the mobile robot 300's communication module and stored in the computer-readable storage medium for processing at a later time based on the schedule or immediately.

In other examples, the mobile robot 300 may emit vaporized disinfectant via various ports or portals. In one particular example, the vaporized disinfectant may be VHP or HPV (hereinafter throughout the disclosure and the drawings that these two abbreviations may be used interchangeably).

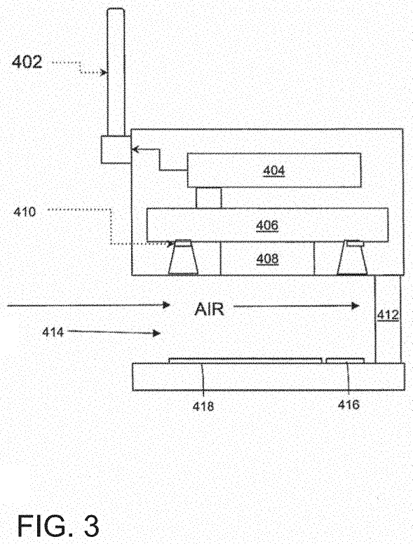

Referring to FIG. 3, in one example, the probe 400 may include an antenna 402, a communication module 404, which may include radio-frequency (RF), wireless, or wireless communication capabilities, a system board 406, an optical sensor 408, a lighting module 410, a fan 412, a compartment 414 and a color chart 416 disposed in the compartment 414. The compartment 414, which is ventilated by fan 412, may be configured to receive data from a chemical indicator 418. The lighting module 410 may include a plurality of light-emitting diodes (LEDs) to illuminate the surfaces of the compartment 414. In another embodiment, the lighting module 410 may be used to illuminate the environments to be disinfected. In yet another embodiment, the lighting module 410 may further provide alert lighting to notify human operators due to exceptions or unexpected event, such as errors, obstacles in the cart/car, etc.

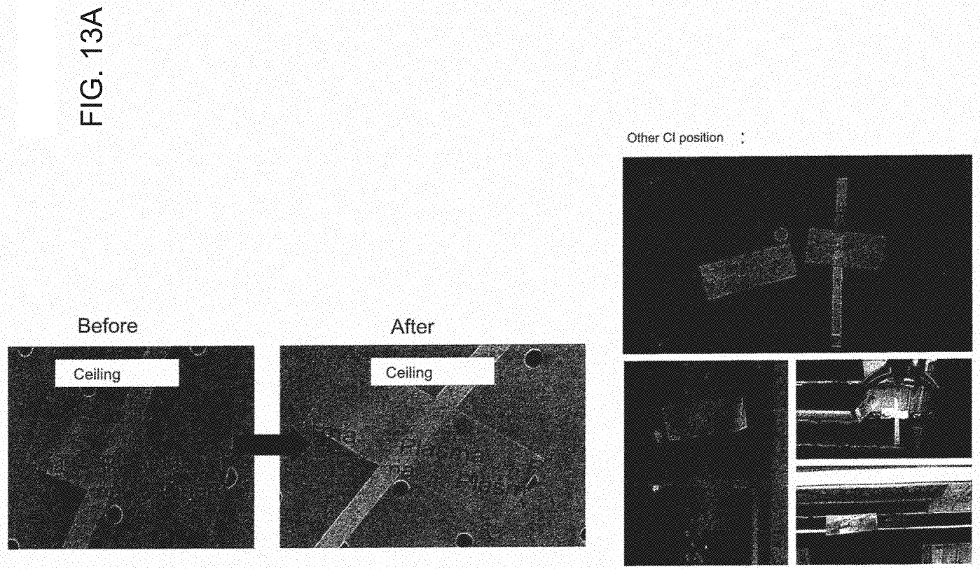

The system board 406 may control the optical sensor 408 to capture at least one image of the chemical indicator 418 with the color chart 416. Similarly, the system board 406 may control the lighting module 410, the fan 412 and the communication module 404 to transmit the image through the antenna 402 to the remote computer system 200 for further analysis. The chemical indicator 418 may undergo color changes according to the concentration of the disinfectant in the environment surrounding it. It is understood that the system board 406 comprises a microprocessor (not shown) and a computer-readable storage medium or memory (not shown) connected to the microprocessor. An exemplary example of a typical use of the mobile robot 300 may be further described in conjunction with FIGS. 12 to 14A-B.

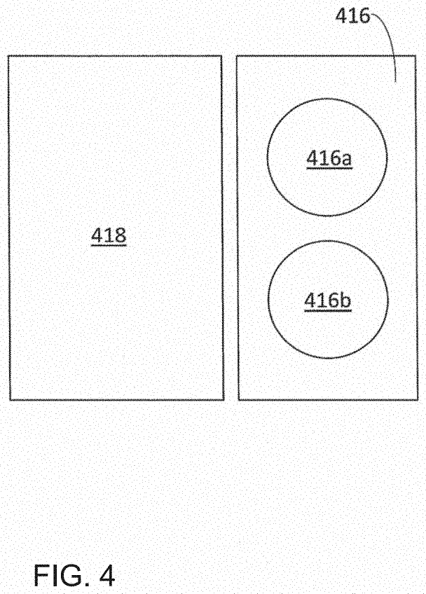

Referring to FIG. 4, a schematic top view of the color chart 416 and the indicator 418. The color chart 416 may include two color spots 416a-416b. In one example, one of them may be pink and the other one may be blue. The chemical indicator 418 may alter or change its color from pink to blue as the concentration of the disinfectant increases. It is to be understood that the color discussed herein is just for illustration purpose. It can be any color and the transition from one color to another may take various forms. It is also to be understood that the chemical indicator 418 may demonstrate the indication by various means. In another embodiment, the spots 416a and 416b may occupy the same position. For example, instead of a discrete placements of the spots 416a and 416b as illustrated, changing of colors may take place in one spot, either 416a or 416b. It is to be understood that other variations of color changing positions or mechanisms may be employed without departing from the spirit and scope of the embodiments of the invention.

In some examples, light module 410 only have one LED.

In some cases, the color chart 416 may have more than two colors.

In some cases, the color chart 416 may have only one color.

In other examples, the color chart 416 may not be affixed to the compartment 414. The color chart 416 may also be integrated to the chemical indicator 418.

In yet another example, the optical sensor 408 may be a CMOS.

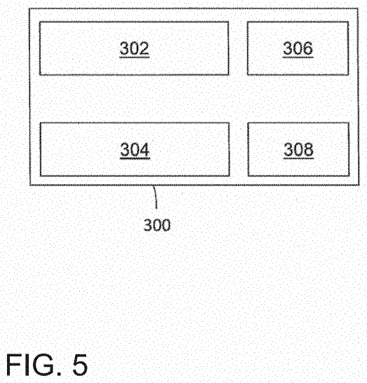

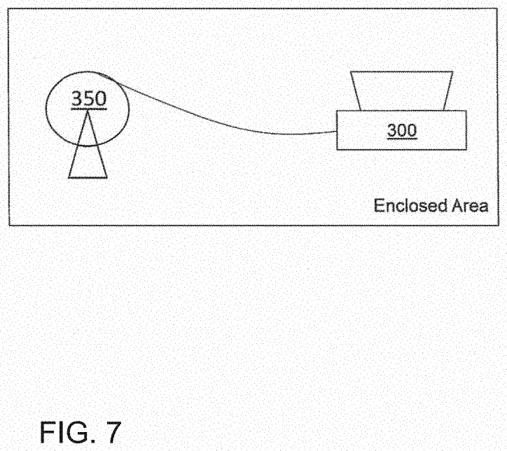

Referring to FIG. 5, the mobile robot 300 may include a disinfectant tank 302, a battery 304, an emitter (e.g., a vaporizer) 306 to vaporize the disinfectant (e.g., VHP) and a portal such as a nozzle/dispenser 308 to dispense the disinfectant to the surrounding environment. The mobile robot 300 also may include one or more spatial sensors to detect its surround environment and at least one wheel to provide actuation to the mobile robot 300. It is also to be understood that the mobile robot 300 may include a motor or a drive (not shown) to drive the mobile robot 300 to move within a spaced area. For example, the mobile robot 300 may also move along a track or have a sensor to enable it to move along a track. For example, the sensor may identify an indicator on the floor to guide the mobile robot 300 to drive along. In another embodiment, the mobile robot 300 may be remote controlled by the user by a joystick or other user interfaces. In yet another embodiment, the mobile robot 300 may be tethered via a tethered cable, such as 350 in FIG. 7, with a separate (e.g., handheld) control unit (not shown). For example, the separate control unit may provide directional instructions. In another embodiment, the tethered cable may also transmit electrical current and data between the control unit and the mobile robot 300. In yet a further embodiment, the drive may be controlled by the disinfection plan downloaded from the remote computer system 200 or the cloud server platform.

In yet another embodiment, the separate control unit may connect to the mobile robot 300 wirelessly and may enable an operator to control the mobile robot 300 remotely. For example, the mobile robot 300 may be coupled to the separate control unit, such as a mobile phone, a tablet, a smartwatch, a smartphone, a bracelet or the like, and the operator may operate the separate control unit to control the mobile robot 300. For example, the separate control unit may include a touchscreen panel illustrating graphical user interface (GUI) enabling the operator to turn on or off the mobile robot 300; to capture image via the probe 400; to move the mobile robot 300 to different locations, etc. In one embodiment, the separate control unit may include a joystick to maneuver the drive or wheel of the mobile robot 300.

In yet another embodiment, the mobile robot 300 may include a GUI thereon so that the operator may control or configure the mobile robot 300 before deployment.

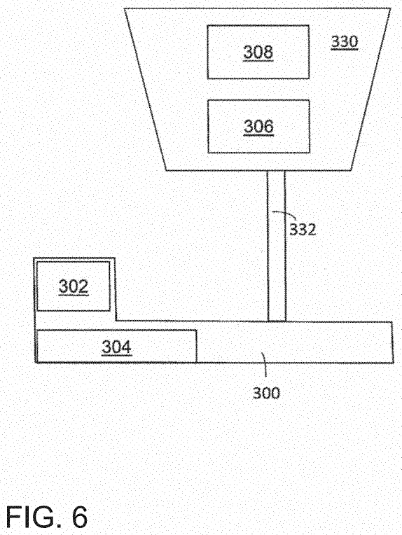

Referring to FIG. 6, the mobile robot 300 may include a separate compartment 330 and a robotic arm 332. The vaporizer 306 and the nozzle/dispenser 308 may be disposed in the compartment 330, which may be extended beyond the main body of the mobile robot 300 by the robot arm 332.

Referring to the FIG. 7, the weight of the mobile robot 300 is reduced by removing its battery 304. A power plug may be disposed on the mobile robot configured to detachably receive a power cable from a retractable power cable reel 350. With this configuration, a single user can hand carry the mobile robot 300 for easy deployment. At the environment of the deployment, the environment may provide a power source for the mobile robot 300. For example, the environment may be a public transportation car or a bus where the floor thereof may provide a panel or track that may provide electric power to the mobile robot 300. The mobile robot 300 may, in this example, include a tether that may engage the panel or track to receive electric power. In another embodiment, the wheel of the mobile robot 300 may include a tether to receive power from the panel or track as it moves along. In yet another embodiment, the mobile robot 300 may include metallic wheels or a metallic “third-wheel” to receive power from the panel or track.

In some examples, the wheel of the mobile robot may be all-wheel drive. In some other cases, the wheel is a continuous track to help the robot 300 to operate on a non-flat handle ground, i.e. door threshold, bumps or ramps.

In yet another embodiment, the wheel of the mobile robot 300 may be composed of continuous tracks so that it may navigate on various terrains. For enclosed area that may have an uneven surface, such as connections between cars or tactile paving for the blind, the mobile robot 300 may still navigate despite the uneven surface. In such an embodiment, the parameters entered above may indicate how different carts/cars are connected, whether there is a door between cars, etc. Based on these parameters, the mobile robot 300 may navigate the cars accordingly.

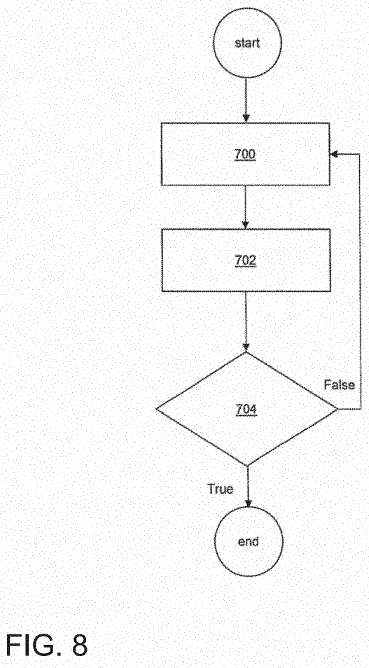

According to another embodiment of the present invention, a method of measuring a concentration of the disinfectant is described. Referring to FIG. 8, at step 700, the probe 400 may capture an image of the chemical indicator 418 with the color chart 416 by the optical sensor 408. The image may then be transmitted to the remote computer system 200 for further analysis in step 702. Color spectrum analysis may be performed on the remote computer system 200 by the control application 202 in step 704. The color spots 416a-416b on the color chart and a predetermined area of the chemical indicator 418 may be located in the image by the control application 202. The spectrum from the first color spot 416a may correspond to the spectrum from the chemical indicator 418 at undesirable concentration of the disinfectant. Similarly, the spectrum from the second color spot 416b may correspond to the spectrum from the chemical indicator 418 at desirable concentration of the disinfectant. The spectrum of each pixel of the predetermined area of the chemical indicator 418 in the image may be compared to the average spectrum from the second color spot 416b. If the number of the pixels in the predetermined area reaching the average spectrum of the second color spot 416b is lower than a predetermined threshold, a false signal may be generated and steps 700, 702 and 704 are repeated. As the air surrounding the probe 400 flow through the compartment 414, as driven by the fan 412, the chemical indicator 418 is continuously exposed to different concentration of the disinfectant. As such, the spectrum from the predetermined area will change as the concentration of the disinfectant in the air increases.

As soon as the number of the pixels in the predetermined area reaching the average spectrum of the second color spot 416b is equal or higher than a predetermined threshold, a positive signal may be generated.

In some examples, the predetermined threshold may be 95% of the total pixels in the predetermined area.

In another examples, the data analysis may be performed at the system board 406 at the probe 400.

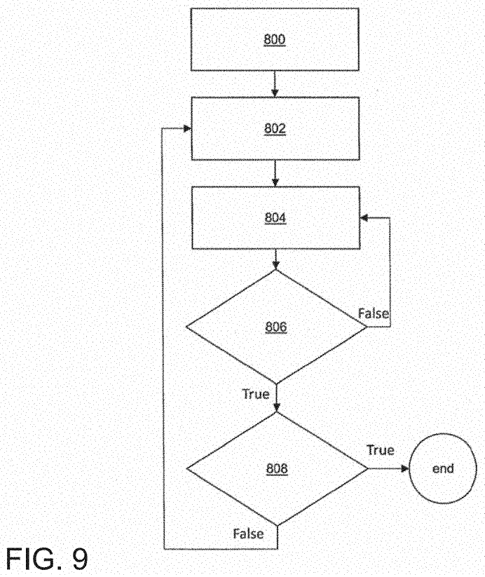

According to another aspect of the present invention, a method of disinfecting the enclosed area may be provided. Referring to FIG. 9, to efficiently clean or disinfect the enclosed area, a number of probes 400 may be installed along a predetermined path of the mobile robot 300. The probes 400 may act as the checkpoints for the mobile robot 300 and provide feedback to the mobile robot 300. At step 800, all probes 400 are initiated and activated. Then the mobile robot 300 may also be activated and may be instructed to generate vaporized disinfectant at the selected checkpoint (for example, the first probe 400 along the path of the mobile robot) at step 802. The mobile robot 300 may stop or hover around the first checkpoint at step 804. At step 806, the remote computer system 200 may determine whether it receives a positive signal from step 704 in the method of detecting a concentration of the disinfectant as discussed above. Once the positive signal is received, the remote computer system 200 may determine whether all the checkpoints have positive signals in step 808. The mobile robot may stop if that condition is met, otherwise, it may instruct the mobile robot to move to the second selected checkpoint and repeat the processes.

In some examples, each checkpoint may have a plurality of probes 400. In this case, step 806 may proceed to step 808 only if a positive signal is received from each of the probes 400.

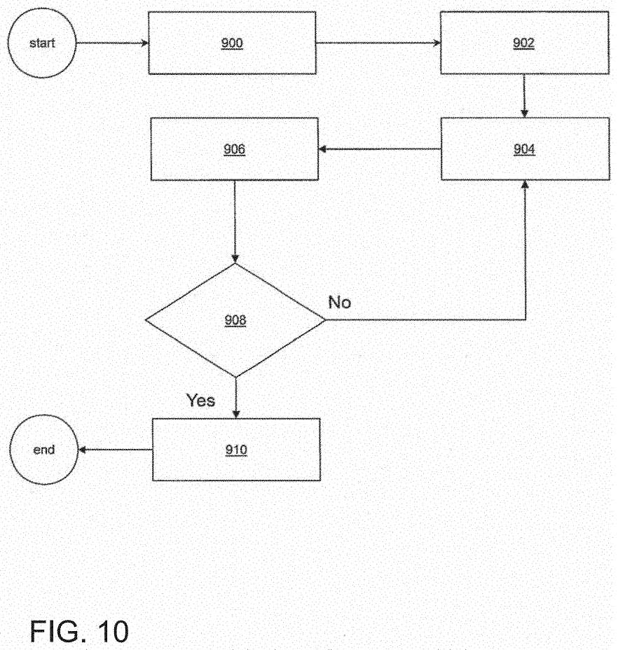

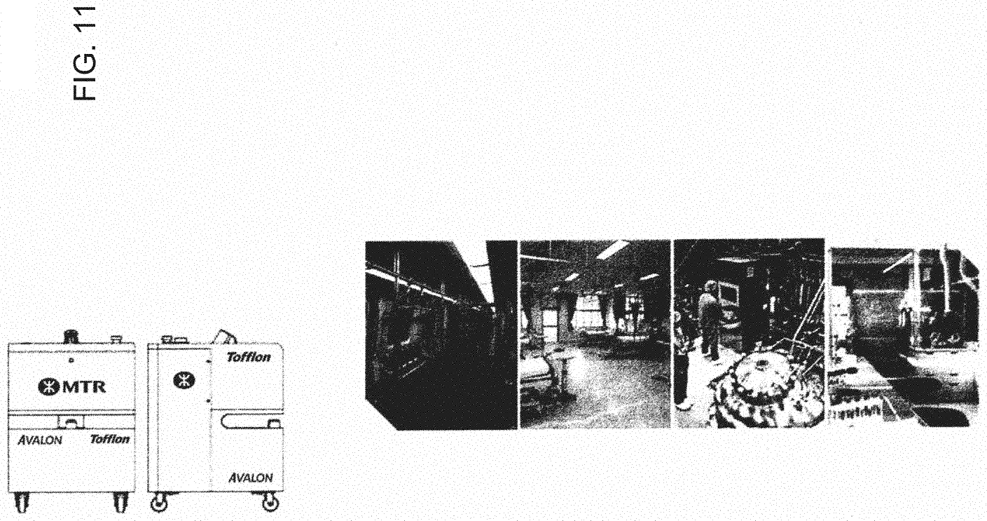

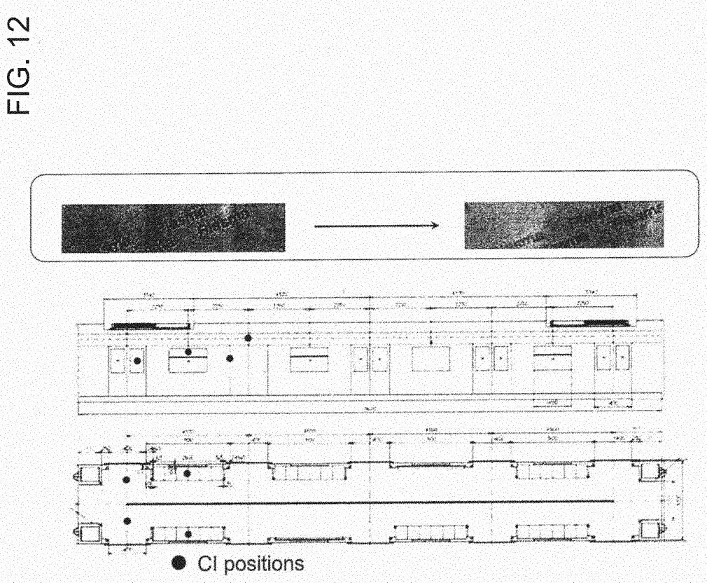

According to another aspect of the present invention, another method of disinfecting the enclosed area is provided. Referring to FIG. 10, a plurality of probes 400 may be disposed in the enclosed area. At step 900, all probes are initiated. Then the vaporizer in the mobile robot 300 is also activated to generate vaporized disinfectant at step 902. The mobile robot 300 may stop or hover around the enclosed area. The remote computer system 200 may continue to fetch the images from all probes 400 at step 904. All images are analyzed in the manner as discussed steps 700, 702 and 704 in step 906. At step 908, the remote computer system 200 may determine if all probes meet disinfectant exposure criteria. The computer system 200 may instruct the mobile robot 300 to stop only if all probes 400 meet the disinfectant exposure criteria in step 910.

In some example, the probe 400 may come with wheels. This mobile probe 400 may move around the enclosed area for measurement as an initial setup. It may be remote-controlled by the control application 202.

In some example, the probe 400 may be equipped with temperature, pressure and related humidity sensors for calibration and logging purpose. It is to be understood that other related sensors may be installed to provide data input to the control application 202 or the remote computer system 200.

In particular, it is advantageous to deploy the present invention in public transport, for example, train, airplane and bus to minimize exposure to healthcare professionals. Of course, these are only an example and is not intended to limit the scope of this patent application.

To further illustrate a deployment of an embodiment of the present invention, FIG. 12 illustrates an exemplary exterior design of the mobile robot 300 and the various environments of the deployment according to one embodiment. For example, the mobile robot 300 may weigh about 70 kg with four motorized wheels. The mobile robot 300, within the weight of about 70 kg, may include a lithium battery, 8 liters of disinfectant, etc. In one embodiment, the disinfectant may be hydrogen peroxide (H₂O₂) with a concentration of 100 to 200 ppm. In one example, the concentration may be used to treat an estimate of a space of 1000 to 1500 m³. In another example, the duration of the battery power may be five hours. It is to be understood that the mobile robot 300 may carry the disinfectant with other concentrations or volume without departing from the spirit and scope of the embodiments. In one example, the mobile robot 300 may be deployed to a train (e.g., subway car, train car, bus, etc.), a hospital, a factory or a lab, a shopping mall, or a public transportation station or train platform.

In another embodiment, the mobile robot 300 may be compartmentalized. For example, the mobile robot 300 may have the compartment 330 with the vaporizer 306 and the nozzle/dispenser 308 in one compartment. In another embodiment, the mobile robot 300 may have the tank 302, the vaporizer 306 and the nozzle/dispenser 308 as one compartment. In a further embodiment, to reduce weight and increase deployment, the moving unit, may include a motor and a set of moving elements or other moving mechanisms (e.g., the wheel), as one compartment. This kind of embodiment, may be useful in rapid deployment in an enclosed area. In one example, the compartment 330 (e.g., with the probe 400, the vaporizer 306, the nozzle/dispenser 308, and the tank 302) may be a portable unit having a handle or a strap for ease of transportation. This embodiment then may enable the compartment 330 to dock with the moving unit before deployment. In yet another example, the compartment 330 may include the vaporizer 306, the nozzle/dispenser 308 and the tank 302, but not the probe 400.

For example, in a public transportation system, an entire train that consists a number of carts, may include a moving unit in each of the carts/cars. These moving units may be battery operated and may be charged in a standby mode. When the disinfection is needed, the separate compartment that includes the tank 302, the HPV, the nozzle/dispenser 308, and the vaporizer 306 may then be connected to or engaged with the moving unit so that the mobile robot 300 may be deployed for disinfection.

In yet another embodiment, the compartment of the tank 302, the HPV, the nozzle/dispenser 308, and the vaporizer 306 as a unit may further be available in each cart/car as an emergency use so that train personnel or conductor may quick disinfect the cart/car at moment's notice once combining the compartment with the moving unit.

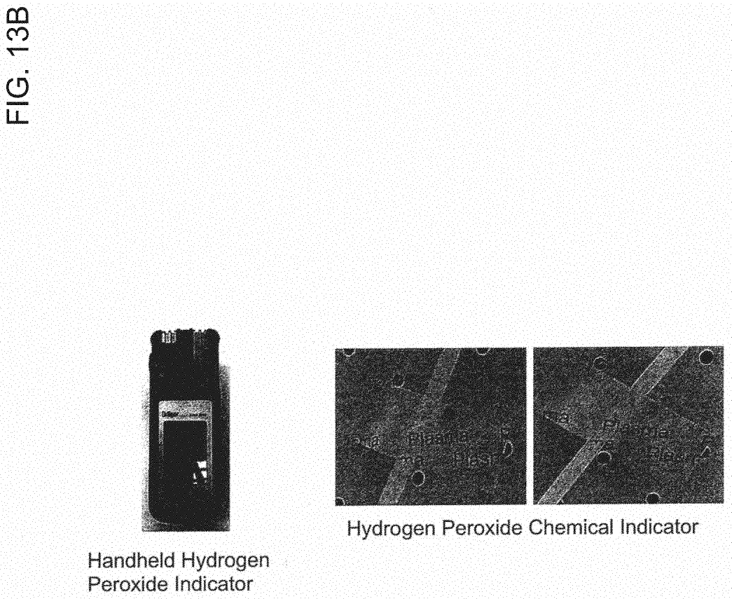

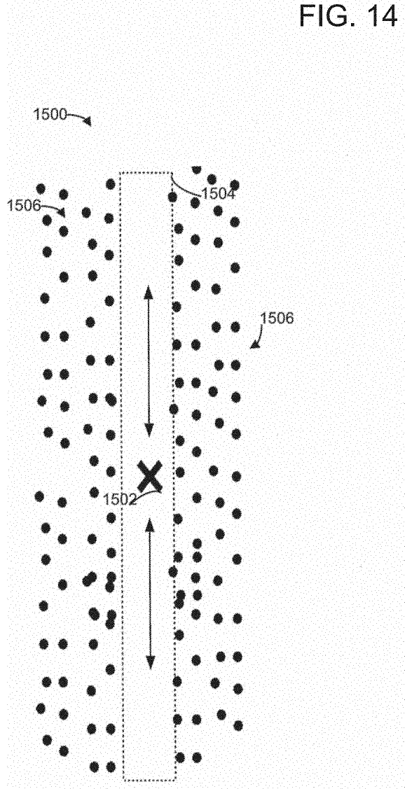

Referring now to FIG. 12, another example of the deployment of the mobile robot 300. In one embodiment, the deployment environment may be a subway car. In such an example, a plurality of probes 400 may be disposed at predetermined locations in the car. Referring now to FIGS. 13A and 14B, the plurality of probes 400 may be disposed on the ceiling of the car, the walls of the car, or other positions of the car. Before the deployment, the probes 400 may be in a non-disinfected state or exhibit a color blue. In one embodiment, the probes 400 may be readily visible to human vision such as shown in FIGS. 13A and 13B. For example, the probe 400 may be a specialized paper that reacts to VHP at a certain concentration. In another example, the probes 400 may not be visible to human vision but may only be visible to the mobile robot 300's optical sensors.

In another embodiment, as shown in FIG. 12, the remote computer system 200, the control application 202, or the cloud server platform may have a visual representation of the locations of the probes 400 within the car so that the users may monitor the progress of the disinfection.

Upon deployment of the mobile robot 300, the mobile robot 300 may begin release of the VHP into the environment (e.g., car as shown in FIG. 13A) to disinfect a portion or a section of the environment. The optical sensors 408, which may already have an approximate location of the probes 400, may monitor and focus the detection of color changes of the probe 400. Upon detecting or sensing the color spectrum change from one color (e.g., blue) to a color designated to be “disinfected” color or state (e.g., pink), the release of the VHP may stop. As discussed above, the mobile robot 300 may then move to the next probe 400.

In another embodiment, the probe 400 may be disposed in a cluster or a set in response to the accuracy range of the sensors 408, either with the assistance of the robotic arm or not. For example, the sensors 408 may have range accuracy that may be calibrated. As such, the probe 400 may be positioned as a function of the desirable accuracy range of the sensors 408, the power consumption of the battery, and the time needed to disinfect or sanitize the environment.

In such an example, once the set of probes 400 has been determined to be disinfected, the mobile robot 300 may move to a next position to disinfect another portion of the environment. The deployment may be completed once the entire environment has been disinfected.

In another embodiment, the remote computer system 200 or the control application 202 may display on a display to show the progress of the disinfection. Alerts may be issued on the control application 202 or the remote computer system 200 to indicate exceptions or notifications with the mobile robot 300. For example, if there is an obstacle on the car, the mobile robot 300 may take a picture or issue an alert and such picture or alert may be transmitted to the remote computer system 200 or the control application 202. In another example, the user may remote control the mobile robot 300 to move away from the obstacle.

In yet another embodiment, more than one mobile robot 300 may be deployed to the same environment at the same time to increase efficiency and reduce time to disinfect. As such, the remote computer system 200 may individually identify each of the mobile robot 300 and the mobile robots 300 may each identify to themselves so that they may sense each other. In term, the user may control the portion(s) or section(s) of the environment each mobile robot 300 may be responsible so that each may be deployed accordingly and may not need to go over to different cars. In such an embodiment, with the probe 400, each mobile robot 300 may still cover the connections between the cars.

In one embodiment where multiple or a plurality of mobile robot 300 may be employed, each mobile robot 300 may be configured to be confined within a given car so it may be more efficient to complete the disinfection of a multiple-car train within a given amount of time.

Referring now to FIG. 14, an illustration shows navigation of the mobile robot 300 in an interior space 1500, such as a public transportation car, a bus, or a ferry. In one embodiment, the mobile robot 300 may include a scanner 1502 that may have a detection range of a medium range (e.g., about 10 or more meters) 3D or 2D scanner. In one embodiment, the scanner may be a light sourced (e.g., laser) or an audio-based (e.g., sonar) scanner to search for an aisle 1504. The mobile robot 300, as described above, may be deployed to mass transportation carrier such as trains, buses, ferries, or aircrafts, etc. These vehicle all share the same characteristics: at least one aisle 1504. Traditional robot use short range detection mechanism such as camera or sonar transducer, etc. The driving logic based on these sensors could be sophisticated yet difficult to fine tune given the amount of time needed to sanitize the cabin may be limited.

The medium range scanner 1502 on the mobile robot 300 may give the mobile robot 300 a 360 degree visibility for 10+ meters. With such rage, the aisle 1504 and its path may easily be recognized in a scanned map like the attached 2D illustration using pattern recognition techniques. As the mobile robot 300 performs a 360 degree medium range scan, coach seats or benches are detected (symbolized by dots in 1506). Since the aisle 1504 is empty, a clear path may be determined in the scanned map. As such, the microprocessor on the mobile robot 300 may use a pattern recognition algorithm to identify the aisle 1504, design a route and energize the motor or driving mechanism of the mobile robot 300 to move along the aisle 1504 accordingly.

In another embodiment, as discussed above, data associated with the aisle 1504, such as the absolute or approximate location or distance thereof, in relation to the seats, benches or even the doors may be downloaded to the microprocessor of the mobile robot 300 so that the mobile robot 300 may supplement its processing as it moves along the aisle 1504.

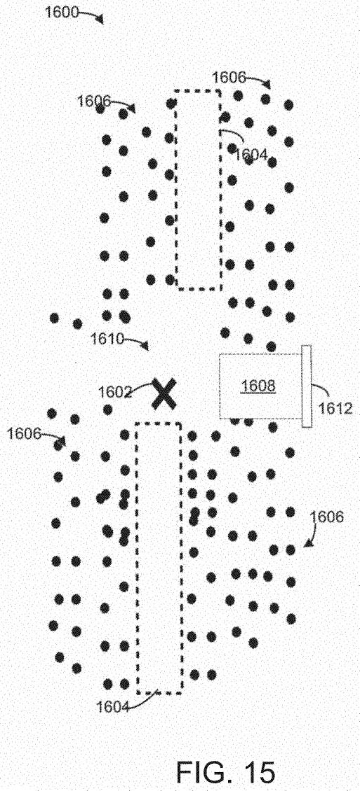

Referring now to FIG. 15, another illustration of the mobile robot 300 in navigating an enclosed space 1600 such as a train or a cabin of an aircraft. In one embodiment, the mobile robot 300 may navigate along an aisle 1604 with a zone or cluster of seats or benches 1606. In this embodiment, the enclosed space 1600 may further include an area 1610 where the mobile robot 300 with a scanner 1602 may need to navigate to the aisle 1604 where there is also a door 1612 nearby.

In such an example, it is not uncommon to have the door 1612. In this illustration, the enclosed space 1600 may be a seat map of a Boeing® 737 aircraft. As illustrated here, there is a short walkway 1608 to the side door 1612. In one embodiment, with the scanner 1602, the aisle detection algorithm may determine the aisle 1604 by a length of the aisle 1604. For example, the aisle 1604 may be longer than the walkway 1608 and as such, the mobile robot 300 would not be confused that the walkway 1608 is the aisle 1604. Similarly, the aisle detection algorithm may confuse the area 1610 may be the aisle based on at least the approach described above.

In one example, the aisle detection algorithm may include parameters such as a minimum length that the aisle is configured to be, height of seats from the floor, etc. Once these parameters are configured, the scanner 1602 may perform the scan and the microprocessor may determine an aisle map that satisfy the parameters. As discussed above, the microprocessor may receive data of the seat map to assist or accelerate the aisle detection. It is to be understood that the seat map may vary from aircraft to aircraft and that airlines may modify the seating arrangements or configurations. As such, the combination of the scanner and the downloaded or loaded information may facilitate the navigation, which may lead to faster or more efficient disinfection.

In yet another embodiment, as the mobile robot 300 moves along the aisle 1504 or 1604, a different nozzle may provide oil-based or water-based antimicrobial solution on the floor (carpet) or seats (e.g., fabric or leather). In such an example, electrostatic spraying may be used. It is to be understood that other means to spray the antimicrobial solution may be provided without departing from the spirit and scope of the embodiments. In another example, a separate tank may be used to hold the antimicrobial solution.

In yet another embodiment, the mobile robot 300 may have one nozzle and, depending on the need, the tank 302 may hold either antimicrobial solution or VHP solution.

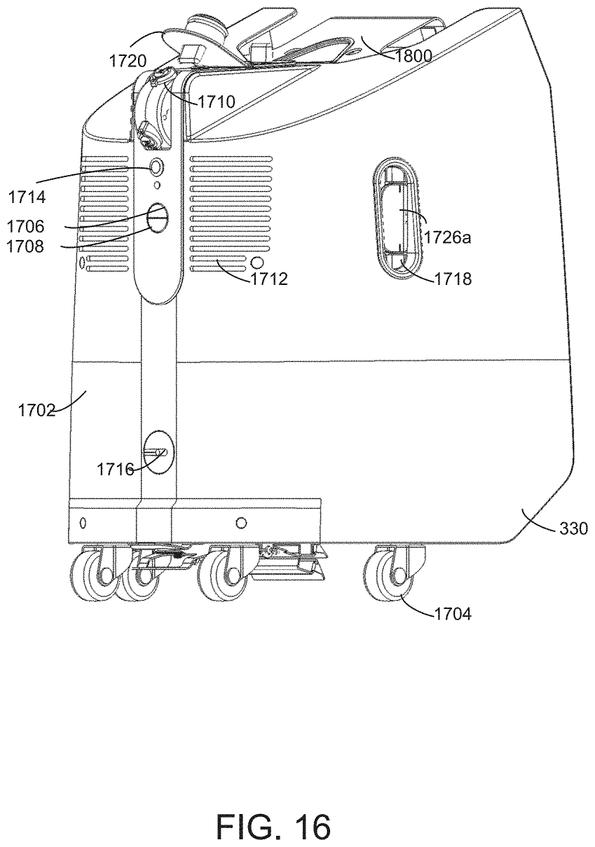

Yet to further illustrate a deployment of some embodiments of the present invention, FIG. 16 illustrates an exemplary compartment 330 configured to detachably dock to a moving unit to form a mobile robot 300. Because of the mobile robot 300, in one embodiment, the compartment 330 may not possess units or elements to drive the compartment 330 on its own. For example, the compartment 330 may not include a driving unit. As such, the compartment 330 may be designed to be attached, engaged with, dock with, or associated with a moving unit, such as the mobile robot 300. In such a configuration, according to some embodiments, the combination of the moving unit or the mobile robot 300 may enhance the capabilities of the moving unit or the mobile robot 300. For example, suppose the mobile robot 300 may have a nozzle to spray in one direction, the compartment 330 may orient its nozzle to spray in an opposite or a different direction. In another example, suppose the mobile robot 300 may include a tank having one type of disinfection formulation or solution for one setting, the compartment 330 may include a solution for another setting or an area so that it reduces the need for switching out the solutions also increase faster deployments. In yet another example, suppose the mobile robot 300 may include a vacuum, sweeping mechanism and/or moping mechanism to clean a surface, the compartment 330 may include nozzle to spray vaporized disinfection solution. Moreover, one moving unit or the mobile robot 300 may be associated with one or more compartments 330 designated for different areas of disinfection.

The compartment 330 may include a housing or a casing 1702 disposed at the front of the compartment 330, a tank for holding antimicrobial solution or hydrogen peroxide solution, at least one motion detector/sensor configured to detect the motion of the compartment 330, at least one battery pack, a plurality of wheels 1704 disposed at the bottom of the compartment 330, an on button 1706 and an off button 1708 disposed at the front of the casing 1702, an nozzle/dispenser 1710 configured to spray vapor disposed at the upper front of the casing 1702, an air ventilator 1712 and a solution level indicator 1714 disposed at the front of the casing 1702; a power socket 1716 configured to receive a power plug to charge the battery pack; at least one handle 1718 configured to easy removal or docking of the compartment 330 from/to the moving unit; and a lid 1720 configured to open or close an inlet of the tank. The compartment 330 may further comprise a detachable mount system 1800 configured to be an mounting interface between the moving unit and the compartment 330.

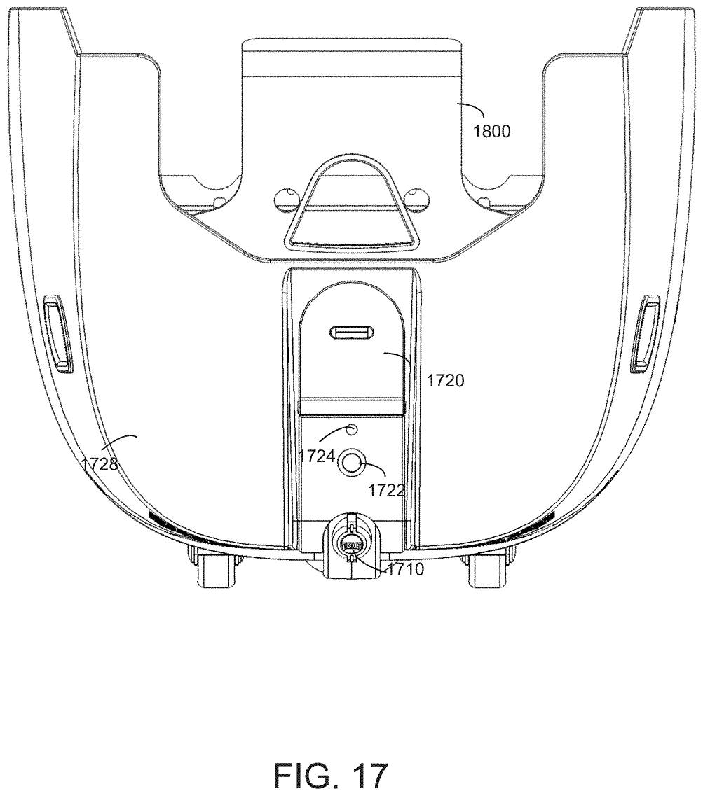

Turning to FIG. 17, the compartment 330 further comprises a nozzle/dispenser power switch 1722 configured to switch on or off the nozzle/dispenser 1710 and a nozzle/dispenser power indicator 1724 configured to indicate whether the nozzle/dispenser 1710 is on or off. FIG. 17 also shows the lid 1720 in its close position. The upper surface 1728 of the casing is inclined from the back to the front of the casing 1702.

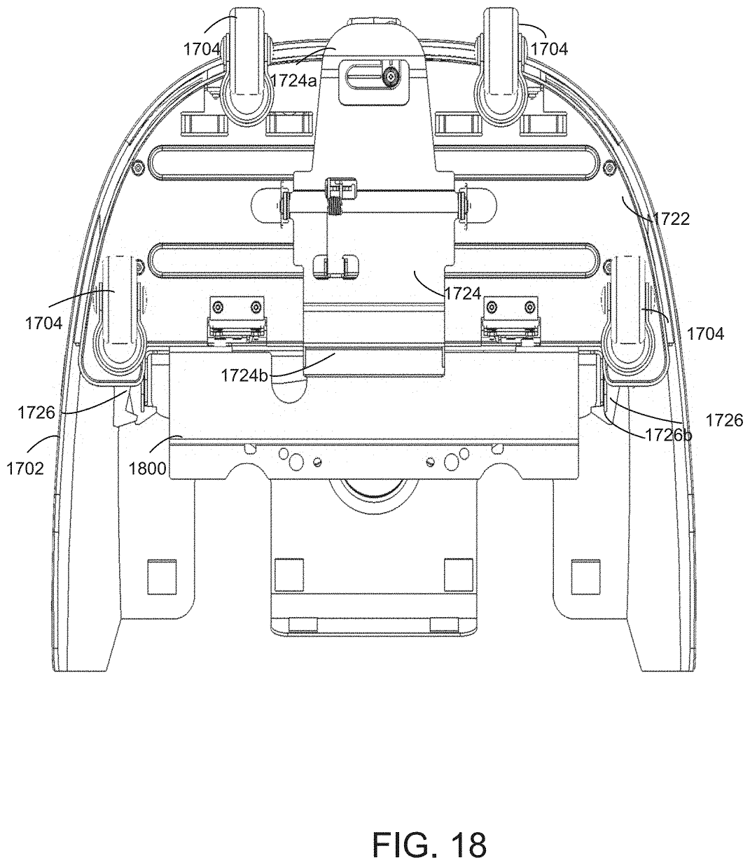

FIG. 18 shows the bottom of the compartment 330, which includes a bottom plate 1722. A bottom mount 1724 is further installed on the bottom plate 1722. The bottom mount 1724 may be a lever having an extended end 1724a extended away from the front of the casing 1702 and a bottom hook end 1724b having a hook structure to hook a bottom bar 1810 of the detachable mount system 1800. The bottom mount 1724 may further comprise a spring configured to push the bottom hook end 1724b towards or engages the bottom bar 1810 of the detachable mount system 1800. The hook structure of the bottom hook end 1724b may release the bottom bar 1810 of the detachable mount system 1800 by pulling the extended end 1724a upward. The hook structure at the bottom hook end 1724b will then be pulled downward and away from the bottom bar 1810 of the detachable mount system 1800, thereby releasing it.

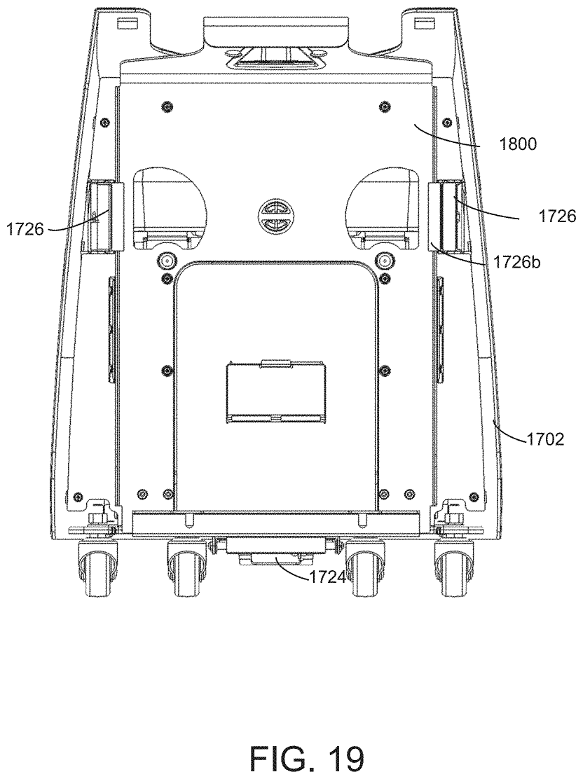

FIG. 19 shows the compartment 330 further comprises two side mounts 1726. The side mounts 1726 are installed together with the handles 1718. Each side mount 1726 may be a lever having a flat end 1726a extended into the handle 1718 and a side hook end 1726b having a hook structure configured to hook a side bar 1808 of the detachable mount system 1800. Each side mount 1726 may further comprise a spring configured to push the side hook end 1726b and its hook structure towards the side bar 1808 of the detachable mount system 1800. The hook structure of the side hook end 1726b may release the side bar of the detachable mount system 1800 by pushing the flat end 1726a of the side mount 1726 towards the handle 1718. The hook structure of the side hook end 1724b will then be pulled away from the side bar 1808 of the detachable mount system 1800, thereby releasing it. In one embodiment, the casing or the body 1702 may include openings for a user to access the handle 1718.

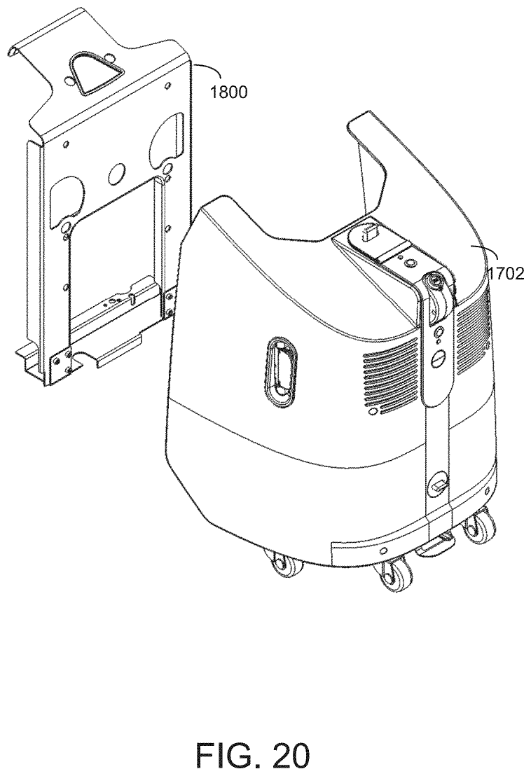

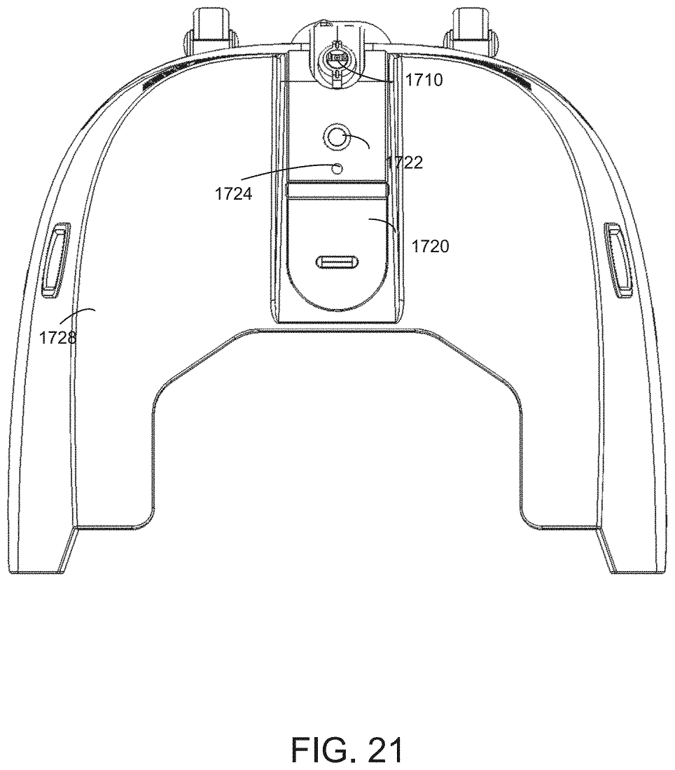

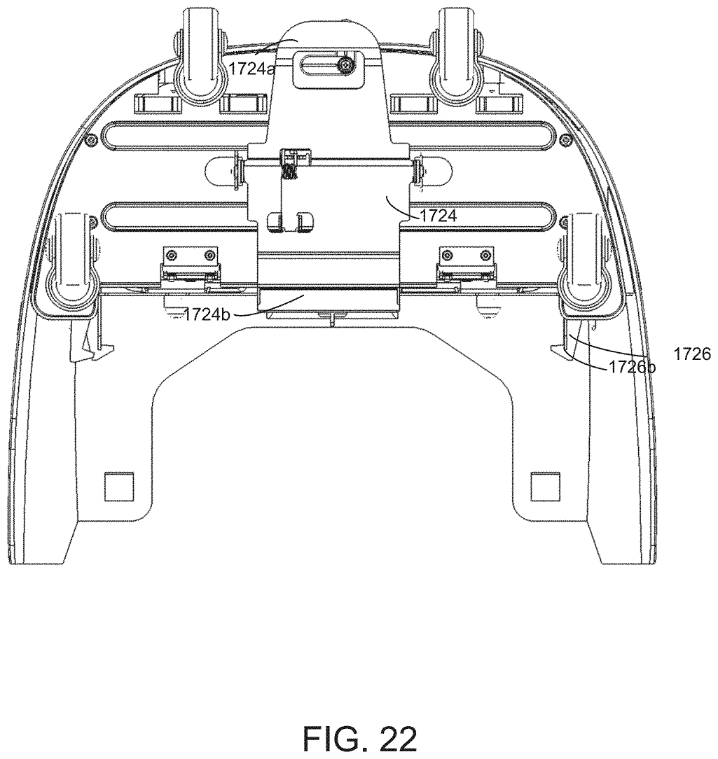

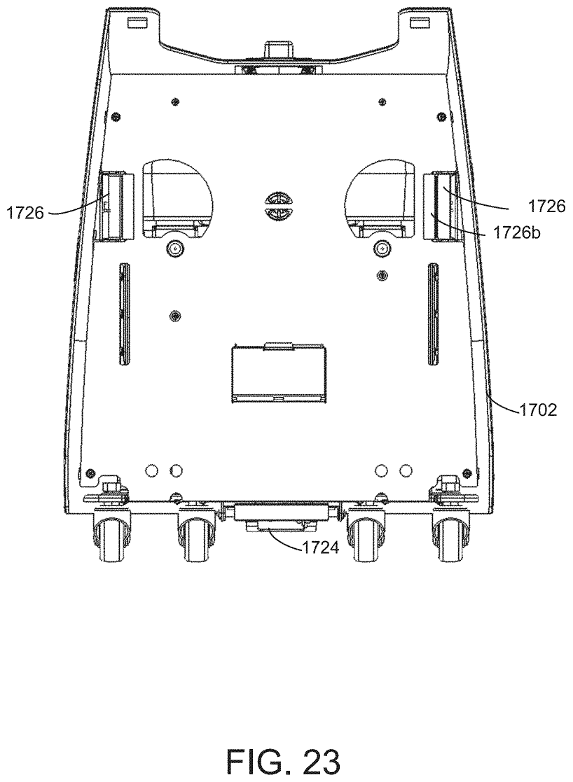

FIG. 20 shows the detachable mount system 1800 being detached from the compartment 330. FIGS. 21-23 shows the top, bottom and back view of the compartment 330 without the detachable mount system 1800 attached thereto.

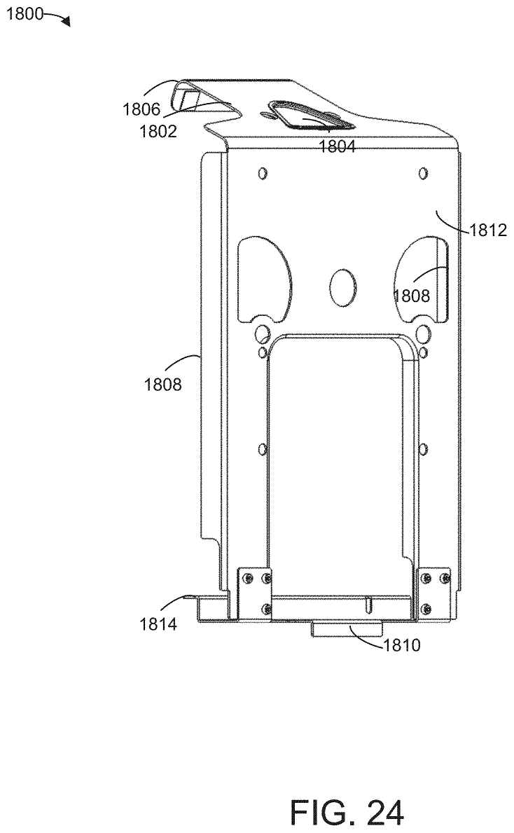

Now turning to FIG. 24, the detachable mount system 1800 includes a neck portion 1802 with a hole 1804 disposed on the neck portion 1802 connecting to a body frame 1812 of the detachable mount system 1800. In one embodiment, the neck portion 1802 further may connect a head 1806 on the other away from the body frame 1812. In one embodiment, the hole 1804 receives a portion or a protrusion of the moving unit in order to secure the detachable mount system 1800 to the moving unit. In another embodiment, the head comprises a hook-like element to further engage a portion of the moving unit. In one embodiment, the head 1806 and the neck portion 1802 form an angle to engage or hook onto a predetermined portion of the moving unit to further secure the attachment of the detachable mount system 1800 to the moving unit. Moreover, the head 1806, the neck portion 1802 and the top of the body frame 1812 may be of contoured shapes to better fitting to portions of the moving unit to provide a streamlined appearance.

The detachable mount system 1800 further includes two side bars 1808 and one bottom bar 1810. The two side bars 1808 may connect on the sides of the body frame 1812. In one embodiment, the side bars 1808 extend a substantial length of the body frame 1812. In another embodiment, the side bars 1808 may extend in a direction toward the head 1806 of the body frame of the detachable mount 1812. In yet another embodiment, the side bars 1808 may extend in a direction that is on the same side of the head 1806 and perpendicular to the plane of the body frame 1812. The bottom bar 1810 may connect to the bottom edge of the body frame 1812 and may protrude downward.

In one embodiment, the bottom bar 1810 and the two side bars 1808 are on different planes, and the bottom bar 1810 is on the same plane as the body frame 1812 of the detachable mount system 1800.The detachable mount system 1800 further includes spacing bar 1814 configured to contact the body of the moving unit at one end and extend the body frame 1812 of the detachable mount system 1800 in the direction toward the head 1806. In one embodiment, the spacing bar 1814 may provide a space between the body frame 1812 of the detachable mount system 1800 or the side bars 1808 and the body of the moving unit.

In one embodiment, the spacing bar 1814 may provide the space needed between the side bars 1808 and the body of the moving unit to enable a secure engagement or contact between the hook structure of the side hook end 1726b and the side bars 1808. In some embodiments, the spacing bar 1814 and the bottom bar 1810 may be attached to the body frame of the detachable mount system 1800 by screws or bolts. In some embodiments, the hole 1804 comprises a shape resembling a triangular.

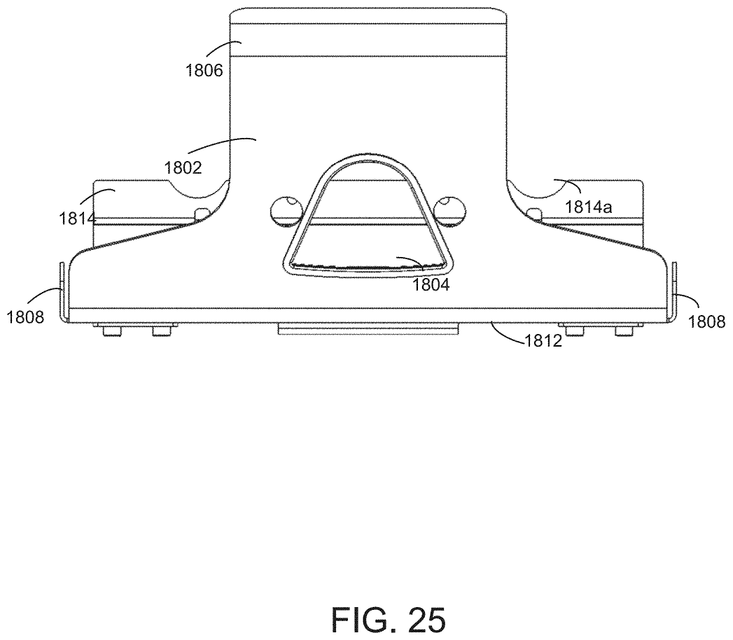

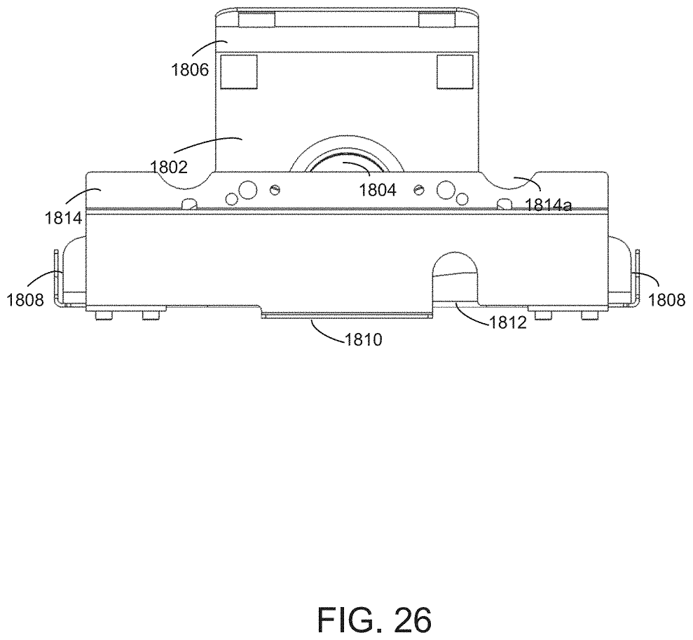

Turning to FIGS. 25 and 26, the spacing bar 1814 may include at least one recess 1814a.

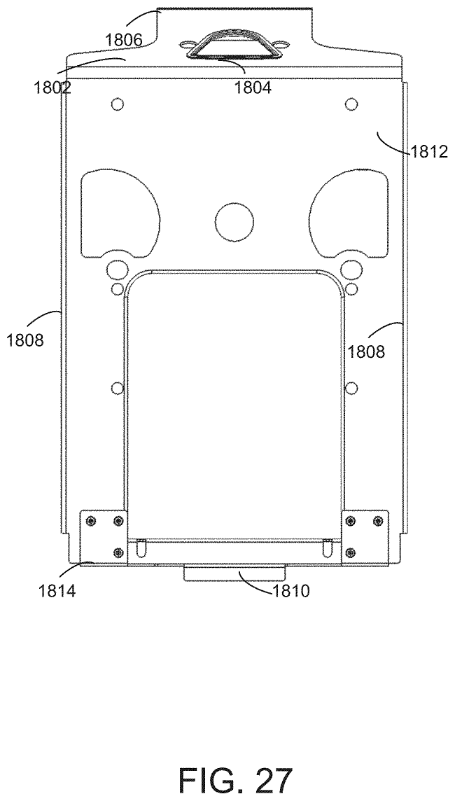

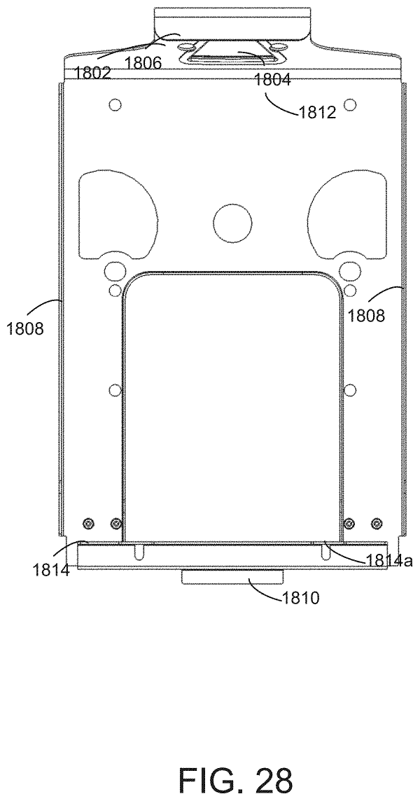

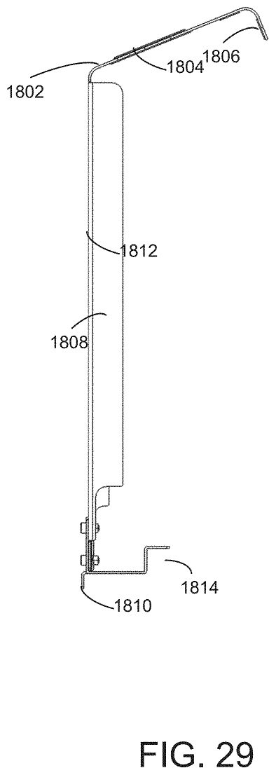

FIGS. 27-39 show the back, front and side view of the detachable mount system 1800 attached thereto.

In some embodiments, the nozzle/dispenser is an electrostatic sprayer or electrostatic gun. Yet in some embodiments, the compartment 330 further comprises more than one tank for holding antimicrobial solution or hydrogen peroxide solution, a vaporizer, and a microcontroller configured to process the data in connection with VPH and/or disinfectant's concentration received, process data received from the motion sensor/detector and control the vaporizer and the nozzle/dispenser 1710. In some embodiments, such data is obtained from the probe 400. In yet some embodiments, the data is obtained from at least one VPH and/or disinfectant's concentration sensor installed on the compartment 330. The solution level indicator 1714 is configured to show the solution level of the at least one tank. In yet some embodiments, the nozzle/dispenser 1710 may further connected to at least one motor such that the pointing direction of the output tip of the nozzle/dispenser can be adjusted by the at least one motor through the instructions it received from the microcontroller. In some embodiments, the compartment 330 further includes a data communication interface to provide a data link between the microcontroller of the compartment 330 and a microcomputer of the moving unit such that the microcontroller may provide input and receive instruction to/from the microcomputer, wherein the input may be concentration of the HPV obtained. The data communication interface may be wired communication interface or wireless communication interface.

In some embodiments, the compartment 330 does not include any driving unit to drive its wheels and cannot move itself.

Now turning to the method of operating the compartment 330. To attach the compartment 330 to the moving unit, the detachable mount system 1800 is first detached from the compartment 330. Thereafter, affix the detachable mount system 1800 to the moving unit by hooking the hook structure 1806 onto a predetermined feature of the moving unit and placing the hole 1804 around a protrusion of the moving unit. Next, move the compartment 330 towards the affixed detachable mount system 1800 and allow the side hook end 1726b of each of the side mounts 1726 to engage the side bar 1808. Similarly, the hook structure of the bottom hook end 1724b of the bottom mount 1724 may engage the bottom bar 1810. Continue to move the compartment 330 towards the detachable mount system 1800 until the hook structure of bottom hook end 1724b and hook structure of the side hook end 1726b hook the bottom bar 1810 and the side bar 1808 respectively.

Upon docking the compartment 330 to the moving unit, it may separately be switched on from the moving unit. Upon switching on the compartment 330, the motion sensor/detector then may detect any motion of the compartment 330 and may feed the signal to the microcontroller of the compartment 330. The microcontroller may control the compartment 330 to spray vaporized disinfectant or VPH when the compartment 330 when the compartment is in motion and stop it from spraying when it cease movement. In some embodiments, the compartment 330 may only spray vaporized disinfectant or VPH after a predetermined amount of time after the compartment 330 is in motion. In some embodiments, the compartment 330 may only stop spraying vaporized disinfectant or VPH after a predetermined amount of time after the compartment 330 comes to at rest. In yet some embodiments, the microcontroller receives spraying instruction from the moving unit through the data link. In another embodiment, some of the elements or embodiments discussed in prior sections with respect to the optical sensors associated with the mobile robot 300 may be added to the compartment 330 as well.

The moving unit comprises at least one wheel, a microcomputer, a driving unit, at least one sensor configured to detecting the surrounding area and provide such data to the microcomputer which may then provide instructions to the driving unit to navigate the moving unit through an area. The sensor may be scanner or any short range detection mechanism as described before. Once the compartment 330 is detachably connected/docked to the moving unit, the moving unit may then carry the compartment 330 as it navigates through the area.

The exemplary embodiments may include additional devices and networks beyond those shown. Further, the functionality described as being performed by one device may be distributed and performed by two or more devices. Multiple devices may also be combined into a single device, which may perform the functionality of the combined devices.

The various participants and elements described herein may operate one or more computer apparatuses to facilitate the functions described herein. Any of the elements in the above-described figures, including any servers, user devices, or databases, may use any suitable number of subsystems to facilitate the functions described herein.

Any of the software components or functions described in this application, may be implemented as software code or computer readable instructions that may be executed by at least one processor using any suitable computer language such as, for example, Java, C++, or Python using, for example, conventional or object-oriented techniques.

The software code may be stored as a series of instructions or commands on a non-transitory computer readable medium, such as a random access memory (RAM), a read only memory (ROM), a magnetic medium such as a hard-drive or a floppy disk, or an optical medium such as a CD-ROM. Any such computer readable medium may reside on or within a single computational apparatus and may be present on or within different computational apparatuses within a system or network.

It may be understood that the present invention as described above may be implemented in the form of control logic using computer software in a modular or integrated manner. Based on the disclosure and teachings provided herein, a person of ordinary skill in the art may know and appreciate other ways and/or methods to implement the present invention using hardware, software, or a combination of hardware and software.

The above description is illustrative and is not restrictive. Many variations of embodiments may become apparent to those skilled in the art upon review of the disclosure. The scope embodiments should, therefore, be determined not with reference to the above description, but instead should be determined with reference to the pending claims along with their full scope or equivalents.

One or more features from any embodiment may be combined with one or more features of any other embodiment without departing from the scope embodiments. A recitation of “a”, “an” or “the” is intended to mean “one or more” unless specifically indicated to the contrary. Recitation of “and/or” is intended to represent the most inclusive sense of the term unless specifically indicated to the contrary.

One or more of the elements of the present system may be claimed as means for accomplishing a particular function. Where such means-plus-function elements are used to describe certain elements of a claimed system it may be understood by those of ordinary skill in the art having the present specification, figures and claims before them, that the corresponding structure includes a computer, processor, or microprocessor (as the case may be) programmed to perform the particularly recited function using functionality found in a computer after special programming and/or by implementing one or more algorithms to achieve the recited functionality as recited in the claims or steps described above. As would be understood by those of ordinary skill in the art that algorithm may be expressed within this disclosure as a mathematical formula, a flow chart, a narrative, and/or in any other manner that provides sufficient structure for those of ordinary skill in the art to implement the recited process and its equivalents.

While the present disclosure may be embodied in many different forms, the drawings and discussion are presented with the understanding that the present disclosure is an exemplification of the principles of one or more inventions and is not intended to limit any one embodiments to the embodiments illustrated.

Further advantages and modifications of the above described system and method may readily occur to those skilled in the art.

The disclosure, in its broader aspects, is therefore not limited to the specific details, representative system and methods, and illustrative examples shown and described above. Various modifications and variations may be made to the above specification without departing from the scope or spirit of the present disclosure, and it is intended that the present disclosure covers all such modifications and variations provided they come within the scope of the following claims and their equivalents.

What is claimed is:

1. A disinfection compartment comprising:
at least one wheel;
a casing disposed on the disinfection compartment;
at least one tank configured to hold antimicrobial solution or hydrogen peroxide solution;
a handle;
a nozzle/dispenser disposed on the casing;
a bottom mount comprising a hook structure at its one end configured to hook a bottom bar of a detachable mount,
a side mount comprising a hook structure at its one end and a flat end,
wherein the hook structure of the side mount is configured to hook a side bar of the detachable mount and the flat end is disposed in the handle, and
wherein the disinfection compartment lacking any driving unit to drive its wheels or move itself.

2. The disinfection compartment of claim 1 further comprising:
an air ventilator disposed at the front of the casing;
a solution level indicator configured to show the solution level of a plurality of tanks;
a battery pack configured to power the disinfection compartment;
a power socket disposed at the front of the casing configured to receive an electric power plug to charge the battery pack;
a motion sensor configured to detect a motion of the disinfection compartment; and
wherein the nozzle/dispenser comprises an electrostatic sprayer or electrostatic gun.

3. The disinfection compartment of claim 2 further comprising:
a motor connected to the nozzle/dispenser configured to adjust the pointing direction of the output tip of the nozzle/dispenser;
at least one sensor configured to detect the concentration of vaporized hydrogen peroxide or disinfectant surrounding the disinfection compartment;
a data communication interface configured to establish a data link with the moving unit; and
a microcontroller configured to receive the concentration data and signal from the motion sensor and connect to the data communication interface.

4. A detachable mount system comprising:
a body frame;
a neck portion having a hole, wherein the neck portion connecting a head and a first end of the body frame;
a plurality of side bars on a side of the body frame, wherein the plurality of side bars extending in a direction toward the head;
a bottom bar disposed at a second end of the body frame; and
a spacing bar extending in the direction toward the head.

5. The detachable mount system of claim 4, wherein the head comprises a hook structure to engage a portion of a moving unit.

6. The detachable mount system of claim 4, wherein the plurality of the side bars engages a portion of a disinfection system.

7. The detachable mount system of claim 4, wherein the hole receives another portion of the moving unit.

8. The detachable mount system of claim 4, wherein the bottom bar engages another portion of the disinfection system.

9. The detachable mount system of claim 4, wherein the spacing bar engages a further portion of the moving unit.

10. A disinfection compartment comprising:
at least one wheel;
a housing;
at least one tank within the housing holding antimicrobial solution or hydrogen peroxide solution;
a handle;
a nozzle/dispenser disposed on the housing; and
wherein the disinfection compartment lacking any independent driving unit to drive its wheels or move itself.

11. The disinfection compartment of claim 10, further comprising:
a plurality of side mounts for engaging a plurality of side bars of a detachable mount system, wherein the detachable mount system engages a portion of a moving unit;
wherein each of the plurality of side mounts comprises a hook and a handle, the handle operably releases or engages the hook to each of the plurality of side bars;
a bottom mount comprising another hook structure to engage a bottom bar of the detachable mount system.
